SEQUENCE LISTING (4 sequences)

SEQ ID NO: 1 - RNA, 791 nt, Homo sapiens
  source 1..791: mol_type = transcribed RNA
agaaagtgctaggcacgtaggcgttcaggagacagtcacggtactcgtttccagacagaagtcatgaggaacaagagggaaggtgctttcccgtgtgcagcgcttggggagactcacacagacagaggatctggcatgacagggaaaggaggaaatggcttctgttaatctctccttcagcttctcccgcccttcccatgcactcttcctgtttccctttccagttctcacggtgactcaaggaacaacgtgtgaaatgaaagacctcaggtgctgtattggctcttgacagctcttcagaagaaaatacctcctgcctgttctgttcagtcctggtgcaggtggtcctggacaacactttgaaaaatgctggggcagaaacaagccagcaagcatatgggattcatctgctgtgagtgataatttatctagtttctaaaaatagtgttataggctggactgtgtccccttcctgtgccccgccgccatgcatatatggacgcctagtacctcagaatgtgactgtatttggaaacagggcctttgaagaagtaattaagttaaaatgaggtcattagggtgggcagtaatacagtataactggtgtccttataagaagagattaggacacagatgcacacagagggaagaccatatgaacacagagggagaagacagccacctgcaagccacagagagagcccttcaaagaaaccaaccctgcagacaatttgatctcaggtttctggcctccagaattgtgagaaaataaatttctgttgtttaagtca

SEQ ID NO: 2 - RNA, 242 nt, Homo sapiens
  source 1..242: mol_type = other RNA
agaaagtgctaggcacgtaggcgttcaggagacagtcacggtactcgtttccagacagaagtcatgaggaacaagagggaaggtgctttcccgtgtgcagcgcttggggagactcacacagacagaggatctggcatgacagggaaaggaggaaatggcttctgttaatctctccttcagcttctcccgcccttcccatgcactcttcctgtttccctttccagttctcacggtgactcaag

SEQ ID NO: 3 - RNA, 99 nt, Homo sapiens
  source 1..99: mol_type = other RNA
gaacaacgtgtgaaatgaaagacctcaggtgctgtattggctcttgacagctcttcagaagaaaatacctcctgcctgttctgttcagtcctggtgcag

SEQ ID NO: 4 - RNA, 450 nt, Homo sapiens
  source 1..450: mol_type = other RNA
gtggtcctggacaacactttgaaaaatgctggggcagaaacaagccagcaagcatatgggattcatctgctgtgagtgataatttatctagtttctaaaaatagtgttataggctggactgtgtccccttcctgtgccccgccgccatgcatatatggacgcctagtacctcagaatgtgactgtatttggaaacagggcctttgaagaagtaattaagttaaaatgaggtcattagggtgggcagtaatacagtataactggtgtccttataagaagagattaggacacagatgcacacagagggaagaccatatgaacacagagggagaagacagccacctgcaagccacagagagagcccttcaaagaaaccaaccctgcagacaatttgatctcaggtttctggcctccagaattgtgagaaaataaatttctgttgtttaagtca

(19) United States
(12) Patent Application Publication
(10) Pub. No.: US 2026/0201372 A1
(43) Pub. Date: Jul. 16, 2026

(54) LONG NON-CODING RNA LIPTER PRESERVES LIPID METABOLISM OF THE HUMAN HEART

(71) Applicant: THE TRUSTEES OF INDIANA UNIVERSITY, Bloomington, IN (US)

(72) Inventor: Lei YANG, Whitestown, IN (US)

(21) Appl. No.: 19/129,942

(22) PCT Filed: Nov. 20, 2023
(86) PCT No.: PCT/US2023/080522

§ 371 Date: May 14, 2025

Related U.S. Application Data
(60) Provisional application No. 63/385,350, filed on Nov. 29, 2022.

(51) Int. Cl. C12N 15/113; A61K 47/69; A61P 3/00; A61P 9/04; C12N 15/86; C12N 15/88; C12Q 1/6809; C12Q 1/6851; C12Q 1/6883

(52) U.S. Cl. CPC C12N 15/113; A61K 47/6911; A61P 3/00; A61P 9/04; C12N 15/86; C12N 15/88; C12Q 1/6809; C12Q 1/6851; C12Q 1/6883; C12N 2310/3231; C12N 2710/10043; C12N 2750/14143

(57) ABSTRACT

Compositions and methods are disclosed for treating metabolic syndrome-associated heart disease cardiomyopathy and/or heart failure, wherein the method comprises the step of increasing the concentration of LIPTER RNA in the cardiomyocytes of said patient.

### CROSS REFERENCE TO RELATED APPLICATIONS

This application claims priority to U.S. Provisional Application Ser. No. 63/385,350, filed Nov. 29, 2022, the contents of which are incorporated herein by reference.

### STATEMENT OF GOVERNMENT SUPPORT

This invention was made with government support under HL160856 awarded by National Institutes of Health. The Government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 2 kilobytes xml file named “391350.xml” created on Nov. 20, 2023.

### BACKGROUND

Metabolic syndrome is defined as the concurrence of obesity-associated cardiovascular risk factors including abdominal obesity, impaired glucose tolerance such as diabetes mellitus, hypertriglyceridemia, decreased HDL cholesterol, and hypertension. Humans suffering from obesity or diabetes mellitus have a higher risk of developing cardiomyopathy or heart failure. Intramyocyte accumulation of lipid droplets (LDs) is associated with cardiomyopathy and heart failure in metabolic disorders characterized by hyperlipidemia, such as obesity and diabetes mellitus. Under hyperlipidemic conditions, LDs accumulate in the myocardium (i.e., cardiac steatosis) and cardiomyocytes (CMs). Although an increased LD level impacts a transient cardiac protective role by storing surplus fatty acids (FAs) in CMs, long-term accumulation of LDs is detrimental, leading to apoptosis, tissue injury and cardiac dysfunction (i.e., lipotoxicity). Examples of metabolic syndrome-associated diseases include, but are not limited to, cancer, cardiovascular disease, diabetes, stroke, hypertension, atherosclerosis, heart failure, concentric cardiac hypertrophy, and myocardial infarction.

Previous studies in animal models also found that an expanded pool of LDs could induce heart dysfunction by impairing cardiac contractility. Currently, the CM intrinsic mechanisms that determine the transition between LD accumulation and mobilization, and the cause of cardiomyopathy in obesity and diabetes mellitus is not well understood, which are critical for investigating potential new therapeutic strategies.

Fatty Acid Oxidation (FAO) occurs when stored LDs are mobilized and transported to mitochondria for β-oxidation. Triacylglycerols (TAG) within the LD core are digested to fatty acids (FAs) via lipolysis and lipophagy processes. The broad interactions of LDs with various organelles, such as the ER, endosomes and mitochondria, facilitate the intracellular lipid trafficking and channeling. LD-associated proteins appear to mediate LD-organelle membrane contacts. For example, FATP1 and DGAT2 at the LD-ER interface regulate LD expansion, whereas LD-mitochondria tethering in skeletal and heart muscle is mediated by PLIN5 for channeling FAs from LDs to mitochondria. Although LD biogenesis, composition and turnover have been extensively studied, the mechanisms that drive intracellular LD transport from the ER to other organelles remain elusive. In human CMs, the intrinsic mechanisms underlying LD transport have not been fully characterized, which is pivotal for FA trafficking and channeling to maintain lipid metabolic balance. A fatty acid oxidation rate is a measure of the rate of oxidation of fatty acids.

Long non-coding RNAs (lncRNAs) are defined as RNAs longer than 200 nucleotides that are not translated into functional proteins. This broad definition encompasses a large and highly heterogeneous collection of transcripts that differ in their genomic origin and functionality. Although RNA mainly functions as carrier and regulator of genetic information, recent evidence indicates that RNA possesses diverse functions via interacting with other molecules, such as nucleic acids, ions, proteins, and lipids. RNA-lipid interactions have shown new regulatory mechanisms in important cellular processes, implying multiplex functional dimensions of RNA.

### SUMMARY

The following disclosure takes advantage of a recent discovery that a human long non-coding RNA (lncRNA), named Lipid-droplets Transfer LncRNA (LIPTER), facilitates lipid droplet (LD) transport within human cardiomyocytes (CMs), thereby preserving lipid metabolism of the human heart. Disclosed herein are compositions and methods to reduce the risk of, or prevent the occurrence of, abnormal lipid metabolism in the human heart, as well as abnormal cardiac lipid metabolism-associated cardiomyopathy, or heart failure in a person diagnosed with or without metabolism syndrome, including obese or diabetic subjects.

In one aspect of the invention, a method of preserving lipid metabolism and function of cardiomyocytes in a patient is disclosed, wherein the method includes enhancing the concentration of LIPTER RNA in said cardiomyocytes. In some embodiments, the method includes increasing the expression of LIPTER RNA in said cardiomyocytes. In certain embodiments, exogenous nucleic acids encoding LIPTER RNA are introduced into said cardiomyocytes. In some embodiments, said exogenous nucleic acids are introduced into said cardiomyocytes via a viral vector, optionally the viral vector is an adeno or adeno-associated viral vector. In some embodiments, said exogenous nucleic acids are introduced into said cardiomyocytes via a nanopartical transfection, optionally wherein nucleic acids encoding LIPTER RNA are bound to the surface of the nanoparticle. In some embodiments, said nanoparticle comprises phosphatidic acid (PA) or phosphatidylinositol 4-phosphate (PI4P). In one embodiment the method includes enhancing the concentration of one or more LIPTER RNAs selected from the group consisting of SEQ ID NO: 1, SEQ ID NO: 2, SEQ ID NO: 3, SEQ ID NO: 4 or fragments thereof in said cardiomyocytes. In one embodiment the method includes enhancing the concentration of one or more LIPTER RNAs selected from the group consisting of any 10, 20, 30, 40, 50 or 100 contiguous nucleotide fragment of SEQ ID NO: 1.

In some embodiments, the concentration of LIPTER RNA in said cardiomyocytes is increased by the introduction of LIPTER RNA into said cardiomyocytes in the form of synthetic locked nucleic acids (LNA), including a locked nucleic acid of any 10, 20, 30, 40, 50, 100 or larger contiguous nucleotide fragment of SEQ ID NO: 1. In one embodiment the introduction of LIPTER RNA into said cardiomyocytes is introduced in the form of synthetic locked nucleic acids (LNA) and comprises the nucleotides of SEQ ID NO: 4). In certain embodiments, said patient is one who has abnormal lipid metabolism of heart and its-associated heart disease, cardiomyopathy, and/or heart failure. In certain embodiments, the patient has cardiac dysfunctions or failure that is associated with metabolic syndromes such as diabetic or obese, or other factors and diseases.

Since LIPTER is significantly down-regulated in subjects with abnormal lipid metabolism of heart tissues, in accordance with one embodiment LIPTER concentrations are used as a biomarker for clinically evaluating the lipid metabolism balance of human cardiomyocytes and heart tissue. Subjects having low LIPTER levels based on comparison to healthy subjects, or average population levels of LIPTER, are identified as subjects with abnormal lipid metabolism. Such identified subjects can be treated with standard therapies for heart disease, optionally including the administration of therapeutic agents to increase LIPTER RNA levels in the subject's cardiomyocytes.

In one aspect, a method of improving fatty acid oxidation, lipid metabolism and heart function in a patient suffering from abnormality of cardiac lipid metabolism-associated heart disease, cardiomyopathy and/or heart failure includes the step of increasing the concentration of LIPTER RNA in the cardiomyocytes of said patient.

Lipid droplets (LDs) are highly dynamic cellular organelles that broadly exist in bacteria to mammalian cells. LDs store excessive lipids to maintain lipid homeostasis. In humans, intramyocyte accumulation of LDs is associated with cardiomyopathy and heart failure in metabolic disorders, including obesity and diabetes mellitus. Although LD-associated proteins are required for LD interactions with other organelles, the direct involvement of RNA in intracellular LD trafficking remains as a mystery. Here, we describe an RNA-mediated lipid droplet transport system in human cardiomyocytes (CMs) via Lipid-droplets Transfer LncRNA (LIPTER; SEQ ID NO: 1). LIPTER directly binds phosphatidic acid (PA) and phosphatidylinositol 4-phosphate (PI4P) on LD surface membrane and non-muscle myosin IIB (MYH10) protein via different domains, which bridges LDs to MYH10-ACTIN cytoskeleton to facilitate LD transport. LIPTER and MYH10 deficiencies in human iPSC-derived CMs (hiPSC-CMs) both result in defective LD transport, mitochondrial dysfunction and increased apoptosis. Conditional Myh10 ablation in mouse CMs promotes LD accumulation, reduces whole heart fatty acid oxidization (FAO) rate, and compromises cardiac functions of mouse hearts. We find NKX2.5 primarily controls CM-specific LIPTER transcription. In vivo, LIPTER transgenic expression enhances whole heart FAO rate, preserves cardiac functions and mitigates cardiomyopathy in both high fat diet-fed and Lepr^{db/db} mouse models. In summary, our findings uncover a molecular linker role of LIPTER RNA in LD transport of human CMs, which is essential for balanced lipid metabolism of the human heart. The present disclosed compositions have broad clinical implications in therapy of lipid metabolism abnormality-associated heart diseases and heart failure.

In accordance with one embodiment, a method of preserving cardiomyocyte function in a patient is provided wherein the expression of LIPTER RNA, or a fragment of LIPTER RNA thereof is increased in the patient's cardiomyocytes. In one embodiment, the patient is one who has lipid metabolism abnormality-associated heart disease, cardiomyopathies and/or heart failure.

Although RNA mainly functions as carrier and regulator of genetic information, recent evidence indicates that RNA possesses diverse functions via interacting with other molecules, such as nucleic acids, ions, proteins, and lipids. RNA-lipid interactions have shown new regulatory mechanisms in important cellular processes, implying multiplex functional dimensions of RNA. Here, we identified a human long non-coding RNA (lncRNA), named Lipid-droplets Transfer LncRNA (LIPTER), in facilitating LD transport within human CMs. LIPTER deficiency impairs LD transport and metabolism, mitochondria function and viability of human CMs. Mechanistically, LIPTER (SEQ ID NO: 1) and biologically active fragments thereof directly binds two phospholipids on a LD membrane, PA and PI4P, and MYH10 motor protein, which connects LDs with the cytoskeleton for intramyocyte LD transport. Importantly, LIPTER overexpression reduces lipid metabolism abnormality, mitigates cardiomyopathy and preserves cardiac functions of obese and diabetic mouse models, suggesting promising clinical potential of LIPTER in treating lipid metabolism abnormality-associated heart disease and failure in human patient. Overall, we provide the first evidence that RNA can directly participate in intracellular lipid transport, and testify the clinical potential of LIPTER in preserving the lipid metabolism, and function of cardiomyocytes in patient with cardiomyopathy and heart failure.

### BRIEF DESCRIPTION OF DRAWINGS

FIG. 1: RT-qPCR detection of LIPTER (LINC00881; SEQ ID NO: 1) expression in human iPSC-derived cardiovascular cell types. As shown in FIG. 1, LIPTER (LINC00881) expression was significantly enhanced in cardiomyocytes (CMs), relative to other cardiovascular cell types. Bars are mean±SD. (n=3). Abbreviations: iPSCs, induced pluripotent stem cells; MCP, multipotential cardiovascular progenitors. (CMs, cardiomyocytes; ECs, endothelial cells; SMCs, smooth muscle cells).

FIG. 2: RT-qPCR results of LIPTER (LINC00881; SEQ ID NO: 1) expression in collected left ventricles of human hearts. RT-qPCR validated that LINC00881 was significantly down-regulated to less than 50% (p<0.05) of that found in non-T2DM hearts. (NF, Non-failure; T2DM, Type 2 diabetes; DCM, Dilated cardiomyopathy).

FIG. 3: Statistical analysis of ratios of cardiac troponin T (cTnT) positive CMs from day 40 of CM differentiation from human iPS cells (hiPSCs). Two LIPTER knockout (LIPTER^{KO}) hiPSC clones were established by using CRISPR/Cas-9, followed by CM differentiation with forming embryoid bodies (EBs). As shown in FIG. 3, significantly reduced CM ratios were detected in LIPTER^{KO} EBs relative to WT EBs at day 40 of CM differentiation. Human iPSC-derived embryoid bodies (EBs). Mean±SD are plotted. *p<0.05 (t-test)).

FIG. 4: Oil Red O and cTnT staining (first two columns) were performed with day 40 human iPSC-derived EBs. LIPTER expression was rescued in LIPTER^{KO} (rescued cells designated: LIPTER^{KO/OE}) hiPSCs by using lentivirus carrying a doxycycline-inducible transgene. As shown in FIG. 4, Oil Red O and cTnT staining of cells revealed that LIPTER KO/OE significantly reduced LD accumulation compared to LIPTER^{KO} hiPSC-CMs (LIPTER^{KO}/OE vs. LIPTER^{KO}). Statistical analysis of the ratio of Oil-red O positive area in cTnT⁺ CM area. Mean±SD are plotted. ***p≤0.001 (t-test).

FIGS. 5A and 5B: FIG. 5A presents data from statistical analyses of maximum respiratory capacity and spare respiratory capacity in OCR. OCR values were normalized to total protein content of each well. Bars are mean±SD. *p<0.05, **p<0.01, (t-test). (n=3).

FIG. 5B presents data from statistical analyses showing the difference of values of maximal oxygen consumption with Palmitate: BSA between without and with Etomoxir (Eto). Compared to WT hiPSC-CMs, mitochondrial maximal respiration capacity, spare respiratory capacity (FIG. 5A) and long chain fatty acid oxidation (FAO) capability of LIPTER^{KO} hiPSC-CMs (FIG. 5B) all were significant reduced. Statistical analyses of ratios of TUNEL⁺ CMs. Bars are mean±SD. *p<0.05, (t-test). (n=3).

FIG. 6: Percentages of Cleaved Caspase-3⁺ CMs in cTnT⁺ hiPSC-CMs. Cleaved-Caspase-3 staining revealed increased apoptosis in LIPTER^{KO} vs. WT hiPSC-CMs, which is reversed by overexpression of LIPTER (LIPTER^{KO/OE}). Bars are mean±SD. *p<0.05, **p<0.01, (t-test).

FIGS. 7A-7C: The gene encoding LIPTER has three exons (exon 1=SEQ ID NO: 2; exon 2=SEQ ID NO: 3; and exon 3=SEQ ID NO: 4). To identify LIPTER domains specifically interacting with PA, PI4P and MYH10, truncated LIPTER fragments were generated as shown in FIG. 7A and used in binding assays. FIG. 7B presents the results of an RNA-lipid overlay assay to detect the binding regions on LIPTER with two phospholipids, PA and PI4P. FIG. 7C presents representative Western blots of MYH10 pulldown by truncated LIPTERs in transfected HEK293T cells using the various truncated LIPTER fragments. (Ab, antibody).

FIGS. 8A-8C: FIG. 8A presents the scheme of a lipotoxicity assay with hiPSC-CMs treated by 400 μM palmitic acid (PA) for 4 days. FIG. 8B presents the data from a statistical analysis of ratios of Cleaved-Caspase3⁺/cTnT⁺ cells in control and LIPTER^{OE} hiPSC-CMs treated with or without 400 μM PA for 4 days. FIG. 8C presents the data from a statistical analysis of ratios of TUNEL⁺ CMs after treatment with 400 μM PA for 4 days. All bars represent mean values±SD. **p<0.01, *p<0.05 (t-test).

FIGS. 9A-9G: FIG. 9A presents the scheme for evaluating effects of LIPTER(Tg) on HFD-induced cardiomyopathy in mice. FIG. 9B presents the data from quantifications of free fatty acid concentrations in mouse hearts after 7-months of HFD feeding. FIG. 9C presents the data from quantifications of TAG concentrations in mouse hearts after 7-months of HFD feeding. FIG. 9D presents the data from baseline FAO rates of whole WT and LIPTER(Tg) mouse hearts at three-month-old age. FIG. 9E provides statistical results of red fibrosis area in whole hearts. FIG. 9F presents data showing left ventricular ejection fraction and FIG. 9G presents data showing left ventricular fractional shortening measurements in WT and LIPTER(Tg) mice fed with HFD for 10 months. All bars represent mean values±SD. **p<0.01, *p<0.05 (t-test).

FIGS. 10A-10E: FIG. 10A presents the scheme for AAV9 delivery of LIPTER and GFP/AS-LIPTER into CMs of Lepr^{db/db} mouse hearts. FIG. 10B presents data from RT-qPCR detection of LIPTER expression in Lepr^{db/db} mouse hearts at 6-weeks post AAV9 injection. FIG. 10C presents data from FAO rates of Lepr^{db/db} mouse hearts at 6-weeks post AAV9 injection. FIG. 10D presents data from left ventricular ejection fraction and FIG. 10E presents data from left ventricular fractional shortening measurements of WT and Lepr^{db/db} mice at 6-weeks post AAV9 injection. All bars represent mean values±SD. **p<0.01, *p<0.05 (t-test).

FIG. 11 provides the RNA sequence of LIPTER (SEQ ID NO: 1).

### DETAILED DESCRIPTION

### Definitions

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

“Subject” refers to any mammal for whom diagnosis, treatment, or therapy is desired including mammals, e.g., humans, laboratory animals (e.g., primates, rats, mice, rabbits and guinea pigs), livestock (e.g., cows, sheep, goats, and pigs), household pets (e.g., dogs, cats, and rodents), and horses.

“Treat,” “treating” or “treatment” refer to an action to obtain a beneficial or desired clinical result including, but not limited to, alleviation or amelioration of one or more signs or symptoms of a disease (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). For example, treating an intracellular pathogen includes decreasing the ability of the pathogen to infect, replicate or maintain viability in a host cell.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

### Embodiments

As disclosed herein, an RNA-mediated lipid droplet transport system has been identified in human cardiomyocytes (CMs) that functions via Lipid-droplets Transfer LncRNA (LIPTER; SEQ ID NO: 1). Without being limited by theory, it is believed that LIPTER directly binds phosphatidic acid (PA) and phosphatidylinositol 4-phosphate (PI4P) on LD surface membrane and non-muscle myosin IIB (MYH10) protein via different domains, which bridges LDs to MYH10-ACTIN cytoskeleton to facilitate LD transport. LIPTER and MYH10 deficiencies in human iPSC-derived CMs (hiPSC-CMs) both result in defective LD transport, mitochondrial dysfunction and increased apoptosis. Conditional Myh10 ablation in mouse CMs promotes LD accumulation, reduces whole heart fatty acid oxidization (FAO) rate, and compromises cardiac functions of mouse hearts. We find that the NKX2.5 gene primarily controls CM-specific LIPTER transcription. This gene encodes a homeobox-containing transcription factor, which is specifically expressed in cardiac progenitor cells and cardiomyocytes, and functions in heart formation and development.

In vivo, LIPTER transgenic expression enhances whole heart fatty acid oxidization (FAO) rate, preserves cardiac functions and mitigates cardiomyopathy in both high fat diet-fed and Lepr^{db/db} mouse models. Accordingly, applicant has uncovered an important role of LIPTER RNA in lipid metabolism of human CMs, which is essential for maintaining balanced lipid metabolism and function of the human heart.

In accordance with one embodiment of the present disclosure, compositions and methods are provided for the treatment of lipid metabolism abnormality associated heart diseases and heart failure. In one embodiment intracellular levels of LIPTER RNA are stabilized or increased in cardiomyocytes of patients suffering from lipid metabolism abnormality-associated heart diseases, including obese and diabetic patients. Increased intracellular levels of LIPTER RNA in cardiomyocytes of patients with lipid metabolism abnormality-associated heart disease will decrease LD accumulation, increase whole heart FAO rate and/or enhance cardiomyocyte function thus improving overall heart function and decrease the risk of heart failure.

In accordance with one embodiment a method of preserving or enhancing function and FAO capacity of cardiomyocytes in a patient is provided wherein the expression of LIPTER RNA, or a fragment of LIPTER thereof is increased in the patient's cardiomyocytes. In one embodiment intracellular concentrations of LIPTER RNA are increased by stimulating the expression of endogenous genes encoding for LIPTER. In one embodiment enhanced production of LIPTER RNA is induced by increasing the intracellular concentration of transcription factors involved in the expression of LIPTER RNA. Alternatively, the regulatory elements of the native LIPTER RNA encoding nucleic acid sequences can be modified or targeted by the addition of enhancer elements or other regulatory elements, or alternative promoters to drive the expression of the native LIPTER RNA coding sequences.

In accordance with one embodiment, intracellular concentrations of LIPTER RNA are increased in cardiomyocytes by transfecting cardiomyocytes in vivo with exogenous nucleic acid sequences that encode LIPTER RNA. Any means of transfecting cells known to those skilling in the art can be used to introduce the LIPTER RNA encoding nucleic acids into cardiomyocytes including but not limited to electroporation, transfection, nanoparticle transmission, viral vectors, direct micro injection, biolistic particle delivery, or the use of non-viral carriers, such as liposomes or exosomes.

In one embodiment, exogenous nucleic acids encoding for LIPTER RNA or a bioactive fragment thereof, (optionally exon 3 of LIPTER RNA) are introduced into cardiomyocytes in vivo via a viral vector, including for example an adenoviral vector or adeno-associated viral vector.

In one embodiment, exogenous nucleic acids encoding for LIPTER RNA or a bioactive fragment thereof, (optionally exon 3 of LIPTER RNA) are introduce into cardiomyocytes via a nanoparticle transfection, wherein nucleic acids encoding LIPTER RNA are bound to the surface of the nanoparticle. In a further embodiment the nanoparticle delivery vehicle comprises phosphatidic acid (PA) or phosphatidylinositol 4-phosphate (PI4P) with the nucleic acids encoding LIPTER RNA, or fragments thereof, are bound to the surface of the nanoparticle. In one embodiment the concentration of LIPTER RNA in cardiomyocytes is increased by the introduction of LIPTER RNA in the form of synthetic locked nucleic acids (LNA), optionally supplemented with the introduction of nucleic acids encoding for LIPTER RNA.

In one embodiment, exogenous nucleic acids encoding for LIPTER RNA or a bioactive fragment thereof, (optionally exon 3 of LIPTER RNA; SEQ ID NO: 4) are introduce into cardiomyocytes via liposome or exosome vesicles, which comprises phosphatidic acid (PA) or phosphatidylinositol 4-phosphate (PI4P).

In one embodiment a method of improving cardiomyocyte function and/or increasing cardiac output and preventing heart failure in a patient having cardiac lipid metabolism abnormality, wherein the method comprises increasing intracellular levels of LIPTER RNA in the cardiomyocytes of the patient. In one embodiment a method of treating a diabetic heart disease is provided wherein the method comprises the steps of comprises increasing intracellular levels of LIPTER RNA in the cardiomyocytes of the diabetic patient.

### Embodiments

In accordance with embodiment 1, a method of preserving cardiomyocyte function in a patient is provided, said method comprising enhancing the concentration of LIPTER RNA in said cardiomyocytes.

In accordance with embodiment 2, the method of embodiment 1 is provided wherein the method comprises increasing the expression of LIPTER RNA in said cardiomyocytes.

In accordance with embodiment 3, the method of embodiment 1 or 2 is provided where an exogenous nucleic acid encoding LIPTER RNA is introduced into the cardiomyocytes of the patient.

In accordance with embodiment 4, the method of embodiment 3 is provided wherein said exogenous nucleic acid is introduced into said cardiomyocytes via a viral vector, optionally wherein the viral vector is an adeno or adeno-associated viral vector

In accordance with embodiment 5, the method of embodiment 3 is provided wherein said exogenous nucleic acid is introduced into said cardiomyocytes via a nanoparticle transfection, wherein nucleic acids encoding LIPTER RNA, and/or LIPTER RNA itself, are bound to the surface of the nanoparticle.

In accordance with embodiment 6, the method of embodiment 5 is provided wherein said nanoparticle comprises phosphatidic acid (PA) and/or phosphatidylinositol 4-phosphate (PI4P).

In accordance with embodiment 7, the method of any one of embodiments 1-6 is provided wherein the exogenous nucleic acid encodes an RNA selected from the group consisting of SEQ ID NO: 1, SEQ ID NO: 2, SEQ ID NO: 3, SEQ ID NO: 4 and fragments thereof.

In accordance with embodiment 8, the method of any one of embodiments 1-7 is provided wherein the exogenous nucleic acid encodes an RNA of SEQ ID NO: 4 or a fragment thereof.

In accordance with embodiment 9, the method of embodiment 9 is provided wherein the exogenous nucleic acid encodes a 100 to 150 fragment of SEQ ID NO: 4, optionally nucleotides 1-142 of SEQ ID NO: 4 or nucleotides 143-251 of SEQ ID NO: 4.

In accordance with embodiment 10, the method of any one of embodiments 1-9 is provided wherein the exogenous nucleic acid encodes a 100 contiguous nucleotide fragment of SEQ ID NO: 1.

In accordance with embodiment 11, the method of any one of embodiments 1-10 is provided wherein the concentration of LIPTER RNA in said cardiomyocytes is increased by the introduction of an exogenous source of LIPTER RNA into said cardiomyocytes, optionally in the form of synthetic locked nucleic acids (LNA).

In accordance with embodiment 12, the method of any one of embodiments 1-11 is provided wherein said exogenous source of LIPTER RNA is introduced into said cardiomyocytes via a viral vector, optionally wherein the viral vector is an adeno or adeno-associated viral vector.

In accordance with embodiment 13, the method of any one of embodiments 1-11 is provided wherein said LIPTER RNA is introduced into said cardiomyocytes via a nanoparticle transfection, wherein nucleic acids encoding LIPTER RNA are bound to the surface of the nanoparticle.

In accordance with embodiment 14, the method of any one of embodiments 1-13 is provided wherein the LIPTER RNA comprises an RNA selected from the group consisting of SEQ ID NO: 1, SEQ ID NO: 2, SEQ ID NO: 3, SEQ ID NO: 4 and fragments thereof.

In accordance with embodiment 15, the method of any one of embodiments 1-14 is provided wherein the LIPTER RNA comprises an RNA of SEQ ID NO: 4 or a fragment thereof.

In accordance with embodiment 16, the method of embodiment 15 is provided wherein the LIPTER RNA comprises a 100 to 150 fragment of SEQ ID NO: 4, optionally nucleotides 1-142 of SEQ ID NO: 4, or nucleotides 143-251 of SEQ ID NO: 4.

In accordance with embodiment 17, the method of any one of embodiments 1-16 is provided wherein the LIPTER RNA comprises a 100 contiguous nucleotide fragment of SEQ ID NO: 1.

In accordance with embodiment 18, the method of any one of embodiments 1-17 is provided wherein said patient is one who has lipid metabolism abnormality in the CMs of heart.

In accordance with embodiment 19, the method of any one of embodiments 1-18 is provided wherein said patient is one who has metabolic syndromes associated cardiac dysfunctions, cardiomyopathy and/or heart failure, including but not limited to diabetes and obesity.

In accordance with embodiment 20, the method of any one of embodiments 1-19 is provided wherein said patient is one who has cardiac dysfunctions, cardiomyopathy and/or heart failure.

In accordance with embodiment 20, a method of improving heart function and/or FAO in a patient suffering from abnormal lipid metabolism of CMs and its-associated cardiac dysfunction, cardiomyopathy and/or heart failure is provided, said method comprising the step of increasing the concentration of LIPTER RNA in the cardiomyocytes of said patient.

In accordance with embodiment 21, a method of improving heart function and FAO in a patient suffering from cardiac dysfunctions, and/or heart failure is provided, said method comprising the step of increasing the concentration of LIPTER RNA in the cardiomyocytes of said patient.

In accordance with embodiment 22, the method of embodiment 20 or 21 is provided where an exogenous nucleic acid encoding LIPTER RNA is introduced into the cardiomyocytes of the patient.

In accordance with embodiment 23, the method of embodiment 22 is provided wherein said exogenous nucleic acid is introduced into said cardiomyocytes via a viral vector, optionally wherein the viral vector is an adeno or adeno-associated viral vector

In accordance with embodiment 24, the method of embodiment 22 is provided wherein said exogenous nucleic acid is introduced into said cardiomyocytes via a nanoparticle transfection, wherein nucleic acids encoding LIPTER RNA are bound to the surface of the nanoparticle.

In accordance with embodiment 25, the method of embodiment 24 is provided wherein said nanoparticle comprises phosphatidic acid (PA) and/or phosphatidylinositol 4-phosphate (PI4P).

In accordance with embodiment 26, the method of any one of embodiments 20-25 is provided wherein the exogenous nucleic acid encodes an RNA selected from the group consisting of SEQ ID NO: 1, SEQ ID NO: 2, SEQ ID NO: 3, SEQ ID NO: 4 and fragments thereof.

In accordance with embodiment 27, the method of any one of embodiments 20-25 is provided wherein the exogenous nucleic acid encodes an RNA of SEQ ID NO: 4 or a fragment thereof.

In accordance with embodiment 28, the method of embodiment 27 is provided wherein the exogenous nucleic acid encodes a 100 to 150 fragment of SEQ ID NO: 4, optionally nucleotides 1-142 of SEQ ID NO: 4 or nucleotides 143-251 of SEQ ID NO: 4.

In accordance with embodiment 29, the method of any one of embodiments 20-28 is provided wherein the exogenous nucleic acid encodes a 100 contiguous nucleotide fragment of SEQ ID NO: 1.

In accordance with embodiment 30, the method of any one of embodiments 20-29 is provided wherein the concentration of LIPTER RNA in said cardiomyocytes is increased by the introduction of an exogenous source of LIPTER RNA into said cardiomyocytes, optionally in the form of synthetic locked nucleic acids (LNA).

In accordance with embodiment 31, the method of embodiment 20 or 21 is provided wherein said exogenous source of LIPTER RNA is introduced into said cardiomyocytes via a viral vector, optionally wherein the viral vector is an adeno or adeno-associated viral vector

In accordance with embodiment 32, the method of embodiment 20 or 21 is provided wherein said LIPTER RNA is introduced into said cardiomyocytes via a nanoparticle transfection, wherein nucleic acids encoding LIPTER RNA are bound to the surface of the nanoparticle.

In accordance with embodiment 33, the method of any one of embodiments 30-32 is provided wherein the LIPTER RNA comprises an RNA selected from the group consisting of SEQ ID NO: 1, SEQ ID NO: 2, SEQ ID NO: 3, SEQ ID NO: 4 and fragments thereof.

In accordance with embodiment 34, the method of any one of embodiments 30-33 is provided wherein the LIPTER RNA comprises an RNA of SEQ ID NO: 4 or a fragment thereof.

In accordance with embodiment 35, the method of embodiment 34 is provided wherein the LIPTER RNA comprises a 100 to 150 fragment of SEQ ID NO: 4, optionally nucleotides 1-142 of SEQ ID NO: 4, or nucleotides 143-251 of SEQ ID NO: 4.

In accordance with embodiment 36, the method of any one of embodiments 30-35 is provided wherein the LIPTER RNA comprises a 100 contiguous nucleotide fragment of SEQ ID NO: 1.

In accordance with embodiment 37, a method of identifying subjects with abnormal lipid metabolism is provided, said method comprising the steps of
measuring LIPTER expression levels in a test subject's cells;
comparing the detected LIPTER expression levels to the levels found in healthy subjects;
identifying a test subject with abnormal lipid metabolism when the detected LIPTER expression levels are lower than the levels found in healthy subjects.

In accordance with embodiment 38, the method of embodiment 37 is provided wherein the LIPTER expression levels are measured by RT-qPCR, optional using RNA recovered from cardiomyocytes of said test subject.

In accordance with embodiment 39, the method of embodiment 37 or 38 is provided wherein a test subject is identified with abnormal lipid metabolism when the detected LIPTER expression levels are at least 5% lower than the levels found in healthy subjects.

In accordance with embodiment 40, the method of embodiment 37 or 38 is provided wherein a test subject is identified with abnormal lipid metabolism when the detected LIPTER expression levels are at least 10% lower than the levels found in healthy subjects.

In accordance with embodiment 41, a method of treating subjects identified as having abnormal lipid metabolism, said method comprising the steps of
measuring LIPTER expression levels in a test subject's cells;
comparing the detected LIPTER expression levels to the levels found in healthy subjects;
identifying a test subject with abnormal lipid metabolism when the detected LIPTER expression levels are lower than the levels found in healthy subjects
treating said identified test subjects using standard therapies for heart disease optionally including the administration of therapeutic agents to increase LIPTER RNA levels in the subject's cardiomyocytes.

In accordance with embodiment 42, the method of embodiment 41 is provided wherein the LIPTER expression levels are measured by RT-qPCR, optional using RNA recovered from cardiomyocytes of said test subject.

In accordance with embodiment 43, the method of embodiment 41 or 42 is provided wherein a test subject is identified with abnormal lipid metabolism when the detected LIPTER expression levels are at least 5% lower than the levels found in healthy subjects.

In accordance with embodiment 44, the method of embodiment 41 or 42 is provided wherein a test subject is identified with abnormal lipid metabolism when the detected LIPTER expression levels are at least 10% lower than the levels found in healthy subjects.

### Examples

### LncRNA LINC00881 (LIPTER) Expression is Restricted in Human Cardiomyocytes

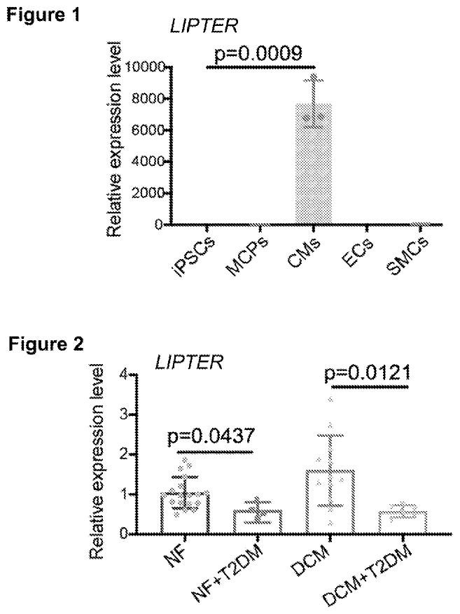

We previously established an in vitro system to differentiate cardiovascular cells from human pluripotent stem cells (hPSCs). By comparing transcriptomic profiles from hPSCs, hPSC-derived multipotential cardiovascular progenitors (MCPs), cardiomyocytes (CMs), smooth muscle cells (SMCs) and endothelia cells (ECs), as well as left ventricle tissues from human with/without type 2 diabetes (T2DM), we identified the top four CM enriched lncRNAs, including LINC00881, TTN-AS1, SLC8A1-AS1 and NAV2-AS2, were downregulated in human T2DM hearts compared to non-T2DM hearts. RT-qPCR validated that in all collected T2DM hearts, only LINC00881 out of four lncRNAs was significantly down-regulated to less than 50% of (p<0.05) of that in non-T2DM hearts. Multiple tissue-specific gene expression databases reveal that LINC00881 expression is restricted in the human hearts. Additionally, by analyzing single cell RNA-seq (scRNA-seq) data from a 6.5-7-week human embryonic heart, we found LINC00881 expression was specifically enriched in the CM clusters that co-expressed CM-marker genes NKX2.5 and cardiac Troponin T (cTnT). LINC00881 expression level in human CMs was comparable to that of NKX2.5, a key cardiac transcriptional factor (TF). LINC00881 is conserved in human and non-human primates, but not across other species. Although three putative ORFs were predicted in LINC00881 by ORF-FINDER, the predicted peptides were not detected by Western blotting or immunostaining using antibodies against FLAG-tag inserted after each ORF, albeit increased RNA level of LINC00881 was detected in transfected 293T cells. Altogether, LINC00881 is highly enriched in human CMs and significantly downregulated in human T2DM hearts (See FIGS. 1 and 2). In this study, since we found LINC00881 was required for lipid droplet transport in human CMs, LINC00881 was renamed Lipid Droplet Transfer LncRNA (LIPTER).

### LIPTER Deficiency Disrupts Lipid Droplet Transport and Metabolism

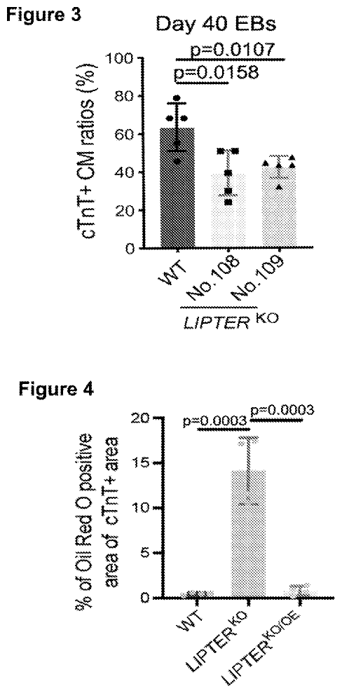

During CM differentiation from hiPSCs, LIPTER expression quickly increased (>10,000 fold, CMs vs. hiPSCs), showing a similar expression dynamic as NKX2.5. To test whether LIPTER affects CM differentiation, two LIPTER knockout (LIPTER^{KO}) hiPSC clones were established by using CRISPR/Cas-9, followed by CM differentiation with forming embryoid bodies (EBs) as we previously described. LIPTER^{KO} did not affect the ratios of beating EBs and cTnT⁺ CMs, nor expression levels of CM markers, cTnT and MYH6, at day 20 of CM differentiation. Since LIPTER expression declines in T2DM hearts, hiPSC-EBs were then cultured for an additional 20 days with high glucose (22.75 mM). FACS detected significantly reduced CM ratios in LIPTER^{KO} EBs than WT EBs at day 40 (See FIG. 3). These data indicate that LIPTER is required for the survival of long-term cultured hiPSC-CMs under high glucose treatment, implying a possible role of LIPTER in CM metabolism. Therefore, untargeted metabolomic analysis was performed with enriched day 40 hiPSC CMs to investigate the global changes of metabolites upon LIPTER deficiency. We found the top 11 increased metabolites in LIPTER^{KO} vs. WT hiPSC-CMs were lipid and lipid-like molecules, including phosphatidylinositol (PI), phosphatidic acids (PA) and triacylglycerols (TAG). Notably, TAG is the main core lipid in lipid droplets (LDs), while PI and PA are phospholipids on the LD membrane. Thus, these data suggested increased amounts of LDs in LIPTER^{KO} hiPSC-CMs than in WT hiPSC-CMs, which was confirmed by Oil Red O and Nile Red lipid staining (LIPTER^{KO} vs. WT; FIG. 4).

Next, we rescued LIPTER expression in LIPTER^{KO} (LIPTER^{KO}/OE) hiPSCs by using lentivirus carrying a doxycycline-inducible transgene. After CM differentiation, LIPTER expression was induced to 2-4-fold more than that of WT hiPSC-CMs. We found LIPTER^{KO/OE} significantly reduced LD accumulation compared to LIPTER^{KO} hiPSC CMs (FIG. 4). All these data demonstrate that LIPTER deficiency disrupts lipid metabolism and causes LD accumulation in hiPSC-CMs.

In CMs, LD accumulation could be caused by increased uptake of free FAs via CD36, a membrane receptor which imports extracellular FAs. However, both CD36 expression and lipid uptake capability was reduced in LIPTER^{KO} hiPSC-CMs compared to WT hiPSC-CMs, indicating that LD accumulation was not caused by enhanced FA uptake. We also detected no significant changes of genes for TAG synthesis and lipolysis on LDs, suggesting that LD accumulation of LIPTER^{KO} CMs may not be primarily due to altered TAG formation or lipolysis process. Interestingly, LIPTER^{KO} reduced expression of PLN5, which was previously reported to mediate LD-mitochondria tethering in CMs. These results implied that LIPTER^{KO} could possibly impair LD transport and/or interaction with mitochondria. To trace LD transport, hiPSC-CMs were cultured with palmitate (200 μM) for 6 h to induce LD formation. In WT hiPSC-CMs, accumulated LDs were observed surrounding nucleus and broadly distributed in cytoplasm, while cytosolic transport/distribution of LDs was retarded in LIPTER^{KO} hiPSC-CMs. We then cultured CMs with depletion of palmitate for additional 12 h to promote mobilization of LDs. LDs in WT hiPSC-CMs were fully mobilized, whereas large numbers of LDs were accumulated in LIPTER^{KO} hiPSC-CMs. Our statistical results show that from 6 h to 18 h, the LD content of WT hiPSC-CMs quickly declined to close to 0, whereas high levels of LDs persisted in LIPTER^{KO} hiPSC-CMs. Since mobilized LDs can transfer stored lipids to mitochondria for β-oxidation, we then conducted live cell imaging to trace the LD-mitochondria interaction. Rhodamine-B labeled palmitic acid (PA^{Rh-B}) was added into CM culture and enriched in LDs after 6 h. In WT hiPSC-CMs, approximately 28% of LD_{S}^{Rh-B} fused with mitochondria and disappeared, while only 8% of LD_{S}^{Rh-B} were fused with mitochondria in LIPTER^{KO} hiPSC-CMs. Finally, we immediately isolated mitochondria from hiPSC-CMs after 2 h of PA^{Rh-B}-treatment and quantified the fluorescence levels. Significantly reduced fluorescence level was detected in LIPTER^{KO} vs. WT mitochondria, indicating that LIPTER ablation reduced PA^{Rh-B} transport to mitochondria. Collectively, these results demonstrate that LIPTER deficiency in human CMs disrupts LD transport and mobilization, leading to extensive LD accumulation.

### LIPTER Deficiency Causes Mitochondrial Dysfunction and Apoptosis of CMs

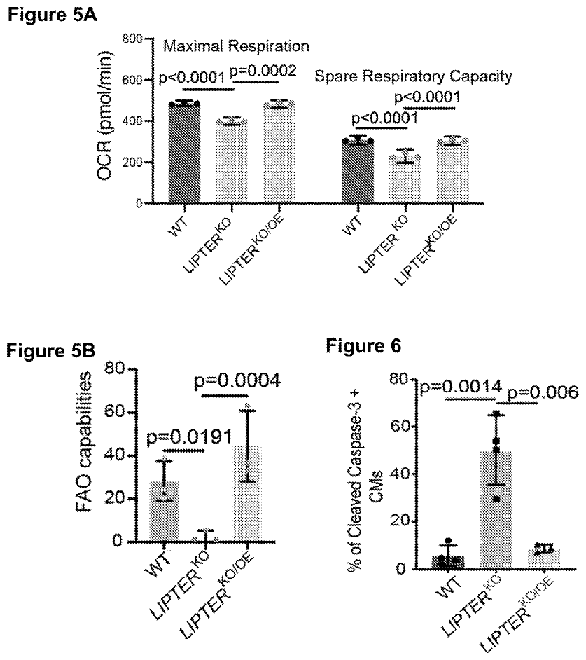

We performed genome-wide mRNA-seq to investigate the global transcriptomic changes in response to LIPTER deficiency. LIPTER^{KO} globally altered transcriptome of hiPSC-CMs, with differentially expressed genes (DEGs) significantly enriched into GO pathways including cellular response to lipid, elevated apoptotic signaling pathway, aberrant mitochondrial functions, and lipid storage, and toxicity related signaling pathways, such as increased heart failure and cardiac dysfunction. Consistent with mRNA-seq results, transmission electron microscopy (TEM) observed compact and rod-shaped mitochondria in WT hiPSC-CMs, whereas ~60% mitochondria of LIPTER^{KO} hiPSC-CMs exhibited giant and/or swelling morphology, which is a hallmark of mitochondrial dysfunction. Compared to WT hiPSC-CMs, mitochondrial maximal respiration capacity, spare respiratory capacity and long chain fatty acid oxidation (FAO) capability of LIPTER^{KO} hiPSC-CMs all were significant reduced (FIGS. 5A and 5B). Increased apoptosis was detected in LIPTER^{KO} vs. WT hiPSC-CMs by performing Cleaved-Caspase-3 staining (FIG. 6). To further study whether these CM abnormalities were LIPTER dependent, we rescued LIPTER expression in LIPTER^{KO} hiPSC-CMs, which restored the swelling morphology and dysfunctions of LIPTER^{KO} mitochondria, and significantly reduced ratios of apoptotic LIPTER^{KO} CMs (FIGS. 5A, 5B and 6).

### NKX2.5 Controls CM-Specific LIPTER Transcription and LIPTER Downregulation in Type 2 Diabetic Heart

Since LIPTER expression is enriched in human CMs and significantly downregulated in T2DM hearts and enhanced LDs were observed in both LIPTER^{KO} hiPSC CMs and T2DM hearts, we posited that LIPTER transcription was under control of a CM-specific regulatory mechanism that could respond to hyperglycemia. By using PROMO, UCSC Genome Browser and TFBIND algorithms, multiple putative binding sites for NKX2.5 and two lipid metabolism related TFs, RXRA and CEBPB, were predicted on LIPTER promoter (~2.5 kb up stream of TSS). Luciferase assays found that NKX2.5 most effectively increased LIPTER promotor activity among the three TFs, and cotransfection of NKX2.5 with RXRA or CEBPB further enhanced LIPTER promoter activities. In WT hiPSC-CMs, NKX2.5 knockdown (NKX2.5^{kd}) by AAV9-shRNAs suppressed expression of LIPTER, but not CM-markers cTnT and MYH10. NKX2.5^{kd} also prominently increased LD accumulation and apoptosis of WT hiPSC-CMs, phenocopying LIPTER^{KO} hiPSC-CMs (shNKX2.5 vs. shControl in WT CMs). All these results indicate that NKX2.5 can control LIPTER transcription in human CMs. Next, we tested the response of NKX2.5-LIPTER axis to hyperglycemia. After treating hiPSC-CMs with high glucose (11.0 and 22.0 mM) for 14 days, LIPTER expression dramatically declined, together with significantly reduced mRNA and protein levels of NKX2.5, RXRA and CEBPB, which is consistent with previous reports that high glucose treatment repressed NKX2.5 and RXRA expressions in mouse hearts. Similarly, reduced NKX2.5, RXRA and CEBPB levels were observed in T2DM hearts when compared with normal human hearts. Finally, we found LIPTER overexpression repressed NKX2.5^{kd}-induced LD accumulation and apoptosis, suggesting the potential of LIPTER in therapy of diabetes associated dilated cardiomyopathy (DCM), which is due to progressive CM loss. Altogether, these data demonstrate that NKX2.5 primarily governs CM-specific LIPTER transcription, and hyperglycemia downregulates the expression of NKX2.5-LIPTER axis in CMs.

### LIPTER Selectively Binds Phospholipids and Non-Muscle Myosin IIB Protein

We next sought to address the molecular mechanisms by which LIPTER regulates LD transport. Colocalization of LIPTER with LDs in CM cytosol was detected by RNA-FISH, suggesting that LIPTER could interact with LDs. We then isolated total lipids, which contain lipid-binding RNAs, from WT hiPSC-CMs, followed with RT-qPCR. Compared to RNA of a house-keeping gene ACTB, LIPTER was highly enriched in isolated lipids (>150-fold, LIPTER vs. ACTB), suggesting that LIPTER could interact with lipid components of CMs. Next, RNA-lipid overlay assay was conducted with LIPTER and antisense-LIPTER (AS-LIPTER), which identified that LIPTER selectively bound phosphatidic acid (PA) and phosphatidylinositol 4-phosphate (PI4P). Since PA and PI4P are located on LD surface membrane and LIPTER is colocalized with LDs, we asked whether LIPTER could bind PA and PI4P in the context of membrane structure. Giant unilamellar vesicles (GUVs) were generated by TopFluor-labeled PI4P or PA and incubated with Alexa-594-labeled LIPTER, respectively. Compared to AS-LIPTER, LIPTER bound PA and PI4P on GUVs. Collectively, these results demonstrate that LIPTER selectively binds PA and PI4P on LD membrane.

Next, the MS2-BioTRAP system was utilized to identify LIPTER-interactive proteins and trace LIPTER in live hiPSC-CMs. After overexpressing 24×MS2-tagged LIPTER and MS2-YFP fusion protein in 293T cells, MS2-YFP protein bound LIPTER-24×MS2 to granulate into green LIPTERMS2-YFP particles in cytosol, which colocalized with red LDs that enriched PA^{Rh-B}. These results are consistent with RNA-FISH results of and confirmed the specific binding of LIPTER-24×MS2 with MS2-YFP protein. Therefore, we pulled down LIPTER-interacting proteins in 293T cells and hiPSC-CMs by using anti-GFP antibody to target MS2-YFP. LIPTER specifically pulled down non-muscle Myosin IIB (NM-IIB, or MYH10), whereas AS-LIPTER did not. RNA-IP results further revealed enrichment of LIPTER by anti-MYH10 antibody in hiPSC-CMs. Confocal immunofluorescent microscopy observed co-localizations of LIPTER with LDs and MYH10 in hiPSC-CM. Live cell imaging revealed that LIPTERMS2-YFP co-localize with migrating LD_{S}^{Rh-B} in hiPSC-CMs. Finally, since myosin motors move along Actin filaments and MYH10 is the only non-muscle myosin in CMs, immunostaining was conducted to observe the MYH10-ACTIN cytoskeleton in hiPSC-CMs. All these results demonstrate that LIPTER can interact with cytoskeleton of hiPSC-CMs via binding MYH10 protein.

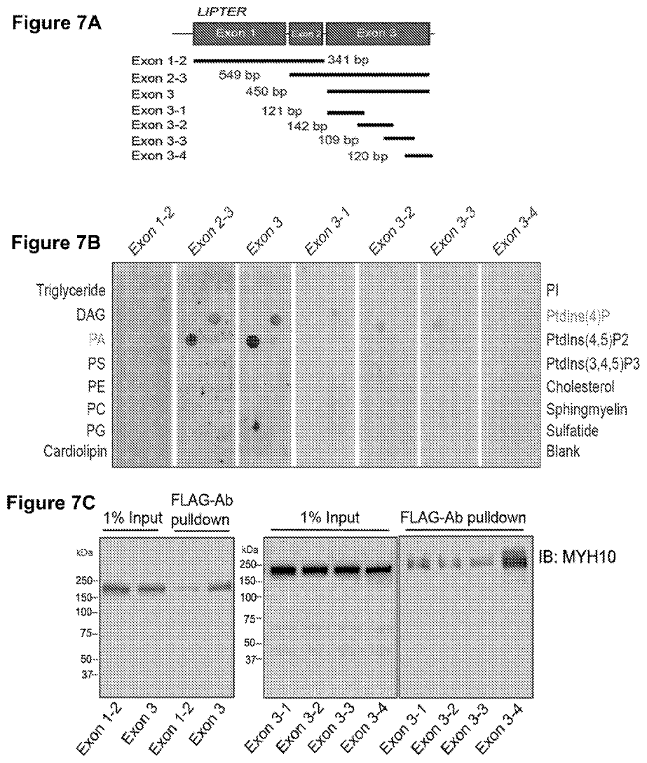

To identify LIPTER domains specifically interacting with PA, PI4P and MYH10, truncated LIPTER fragments were generated (FIG. 7A). GUVs assay was conducted with LIPTER fragments and identified that Exon 3 of LIPTER contained binding domains with PA and PI4P. RNA-lipid overlay assay further detected PI4P binding domain on Exon 3-1, and PA binding domain on both Exon 3-2 and Exon 3-3 regions (FIG. 7B). A MYH10 protein binding domain was identified on Exon 3-4 region by using anti-MYH10 antibody to pull down truncated LIPTER fragments from transfected cells (FIG. 7C). These results demonstrate LIPTER selectively binds PA, PI4P and MYH10 protein via different RNA domains on the Exon 3. Collectively, our data support that LIPTER functions as a molecular linker to connect LDs with cytoskeleton for intramyocyte LD transport.

### Loss-of-MYH10 Phenocopies LIPTER Deficiency in Cardiomyocytes

To further investigate the role of LIPTER-MYH10 interaction in LD transport, MYH10 was knocked out (MYH10^{KO}) in hiPSCs by CRISPR/Cas-9. Compared to WT hiPSC-CMs, MYH10^{KO} hiPSC-CMs showed increased LDs, ~50% of swollen mitochondria, reduced mitochondrial spare respiratory and maximal respiration capacities and FAO capacity, increased apoptosis, retarded LD transport in cytosol, reduced LD fusion with mitochondria together with less PA^{Rh-B} transferred to mitochondria, all of which phenocopied LIPTER^{KO} hiPSC-CMs. Additionally, (S)-(−)-Blebbistatin, which is a chemical inhibitor of ATPase activity of MYH10, treatment enhanced LD accumulation and ratios of TUNEL⁺ CMs compared to WT hiPSC-CMs treated with its inactive enantiomer control, (R)-(+)-Blebbistatin. All these results demonstrate that functional loss of MYH10 could phenocopy LIPTER deficiency in hiPSC-CMs. Next, we asked whether MYH10 inhibition could ablate LIPTER gain-of function. Compared to (R)-(+)-Blebbistatin, (S)-(−)-Blebbistatin enhanced LD accumulation and apoptosis, whereas reduced FAO of LIPTER overexpressing (LIPTER^{KO}/OE) hiPSC-CMs. These results demonstrate the indispensable role of MYH10 in executing LIPTER function. Unlike LIPTER, MYH10 is highly conserved across human and mouse. Therefore, Myh10 was conditionally knocked out in mouse CMs by crossing Myh10^{f/f} with Tnnt2^{Cre} mice (Myh10^{CKO}). After fed with high-fat diet (HFD, 45 kcal % Fat) for 3 months, Myh10^{CKO} mouse hearts exhibited severe lipid accumulation, including elevated LD deposition in CMs and enhanced TAG and FAs levels than Myh10^{f/f} littermate control hearts. Notably, Myh10 deficiency significantly reduced the whole heart FAO rates of HFD-fed Myh10^{CKO} mice compared to those of HFD-fed Myh10^{f/f} hearts. Additionally, cardiac functions, including ejection fraction (EF) and fractional shortening (RS) values, significantly declined in Myh10^{CKO} hearts compared to those of Myh10^{f/f} hearts. Collectively, all these in vitro and in vivo results demonstrate a conserved and critical role of Myh10 in lipid balance of mammalian heart muscle cells.

Gain-of-LIPTER Mitigates Lipotoxicity of hiPSC-CMs

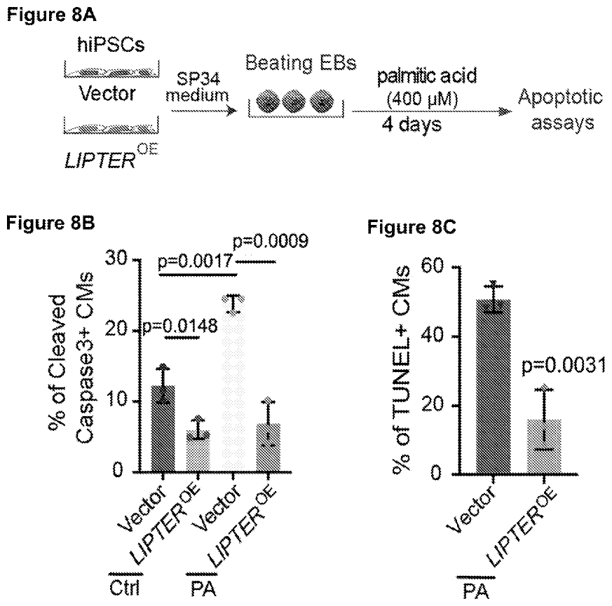

Palmitate treatment was used to mimic lipid-overload, which compromised mouse heart function and increased CM death. In cell culture models of lipotoxicity, palmitate overload incited oxidative and ER stress in CMs (FIG. 8A). We found palmitic acid (PA, 400 μM) treatment for 4 days significantly induced apoptosis of hiPSC-CMs, which was prominently reduced by LIPTER^{OE} (FIGS. 8B, 8C). These data reveal a protective role of LIPTER against lipid-overloaded induced lipotoxicity of hiPSC-CMs.

### In Vivo LIPTER Transgene Ameliorates Obesity and Diabetes-Associated Cardiomyopathy in Mouse Models

CMs within obese and diabetic human hearts exhibit abnormal lipid metabolism, characterized by the high level of LD accumulation when cardiac function is normal, indicating that lipid metabolic disturbance precedes the onset of cardiac dysfunction. Therefore, we posited that gain-of-LIPTER could ameliorate lipid metabolism abnormality-associated cardiomyopathy in obesity and diabetes and prevent subsequent cardiac dysfunction.

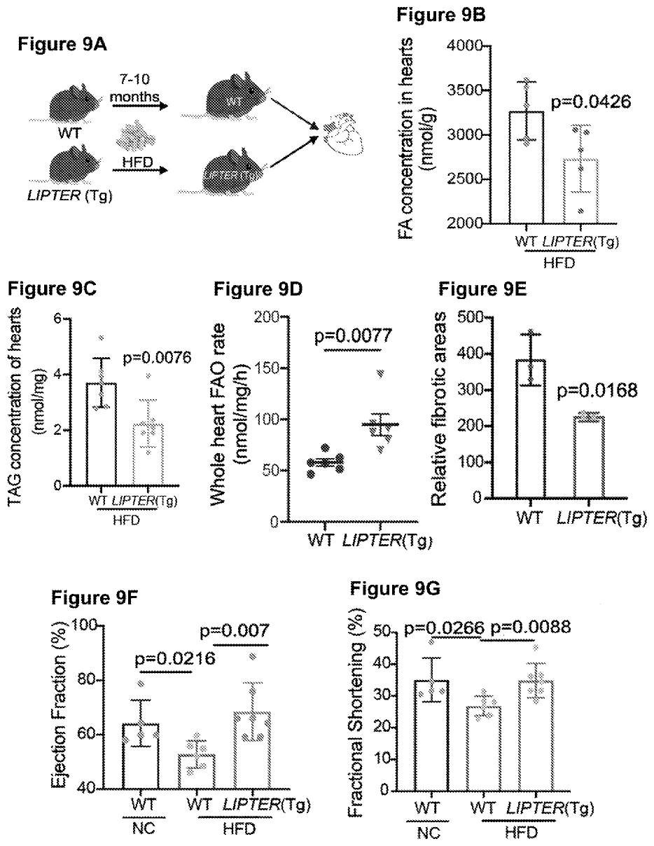

To test this, a LIPTER transgenic (LIPTER(Tg)) mouse line was generated by knocking in LIPTER into the Rosa26 locus. Six-weeks-old LIPTER(Tg) and WT mice were fed with HFD to induce LD accumulation, obesity, insulin resistance and cardiac abnormalities (FIG. 9A). After 7-month-of HFD feeding, LIPTER(Tg) mice showed no significant changes of heart weight/tibia length ratio compared to WT mice. However, LIPTER(Tg) heart had significantly reduced LDs, and FAs and TAG concentrations when compared with similarly treated WT mouse hearts (FIGS. 9B, 9C).

Notably, LIPTER(Tg) prominently enhanced the baseline FAO rates of whole mouse hearts (FIG. 9D). Additionally, significantly reduced fibrotic areas were observed in HFD-fed LIPTER(Tg) hearts compared to WT hearts. Importantly, LIPTER(Tg) preserved cardiac functions of HFD-fed mice. Significantly reduced EF and FS values were observed in WT mice fed with HFD for 10 months compared to WT mice fed with normal chew (NC) (FIGS. 9F, 9G). However, EF/FS values of HFD-fed LIPTER(Tg) mouse hearts were significantly higher than those of HFD-fed WT mice and were preserved at the similar level of WT mouse hearts fed with normal chew (FIGS. 9F, 9G). All these results demonstrate that gain-of-LIPTER can ameliorate HFD-induced cardiomyopathy and preserve cardiac function.

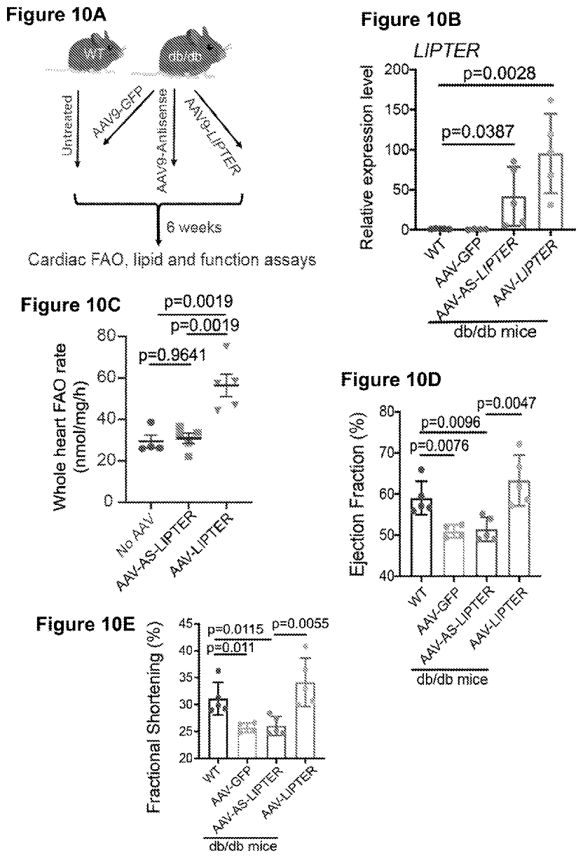

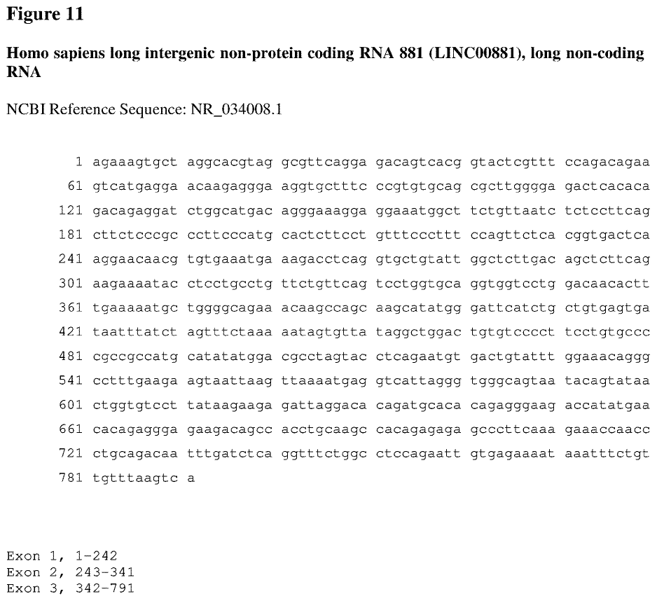

Finally, we investigated the impact of CM-specific LIPTER transgene on cardiomyopathy of leptin receptor deficient Lepr^{db/db} mouse, which is a mouse genetic model of type 2 diabetes and obesity that develops cardiac hypertrophy and dysfunction from 10-weeks of age (FIG. 10A). Adeno-associated virus 9 (AAV-9) carrying a chicken cTnT promoter was utilized for exclusive LIPTER delivery into CMs. We tested the feasibility by retro orbital injection of AAV9-cTnT-GFP virus (2×10¹⁰ vg/g) into WT mice and found highly efficient delivery of GFP in mouse CMs. Next, AAV9-LIPTER and its controls including AAV9-GFP and AAV9-AS-LIPTER viruses were delivered into 6-week-old B6.BKS(D)-Lepr^{db/db} mice via same approach. Age-matched WT C57BL/6J mice without AAV-9 injection were also included as control. Six-weeks post AAV-9 injection, transgenic LIPTER and GFP expressions were detected in mouse hearts (FIG. 10B). AAV9-LIPTER significantly reduced intramyocyte LD accumulation, increased whole heart FAO rate (FIG. 10C), reduced CM size, and preserved cardiac EF and FS values of Lepr^{db/db} mice (FIGS. 10D, 10E) compared to control groups. Since AAV9-LIPTER transgene into mouse CMs did not affect blood glucose level of Lepr^{db/db} mice, all these results indicate that CM-restricted LIPTER transgene expression can mitigate cardiomyopathy and preserve cardiac functions of Lepr^{db/db} mice.

### DISCUSSION

Balanced LD metabolism and handling are critical for lipid homeostasis of CMs and heart function. In this study, we discover and characterize a lncRNA LIPTER-dependent LD transport system in human CMs. Our data indicate that LIPTER functions as an RNA linker to connect LDs with the cytoskeleton thereby facilitating LD transport, which plays an important role in maintaining lipid metabolism balance, mitochondrial function and survival of human CMs.

We find LIPTER binds both PA and PI4P, which are important in membrane-membrane interactions to facilitate LD transfer. Particularly, PA on the ER membrane contributes to newly formed LDs, while PI4P on Golgi and plasma membrane can recruit and bind lipid transport carrier proteins on LDs. Our MS study did not detect typical LD associated proteins pulled down by LIPTER, except TFG (Trafficking From ER To Golgi Regulator). However, gel shift assay did not detect interaction between TFG and LIPTER. These results, together with the GUVs results, indicate that LIPTER can directly bind PA and PI4P without protein factors. Currently, what determines the selective interactions of RNA with different lipids remains unclear. A few studies suggested that RNA-lipid interaction might highly depend on the length, base pairing and nucleotide content of RNA. In particular, Tomasz et al. reported that guanine and G quadruplex formation are critical for RNA-lipid interaction.

We find LIPTER interacts with MYH10 protein to bridge LDs with cytoskeleton for LD transport. Depletions of MYH10 disrupted LD metabolism and transport in hiPSC-CMs and compromised cardiac function of Myh10^{CKO} mice. These data suggest a crucial and conserved role of cytoskeleton in LD trafficking of mammalian heart muscle cells.

A previous study found an 85-fold increase in apoptosis and 4-fold increase in necrosis of myocytes from diabetic hearts compared to non-diseased hearts. Diabetic cardiomyopathy is known as a non-ischemic form of dilated cardiomyopathy (DCM), which is characterized by progressive heart muscle loss, global systolic dysfunction and heart failure. It was reported that approximately 75% of patients with idiopathic DCM were found to be diabetic. Our data reveal that hyperglycemia suppresses the NKX2.5-LIPTER axis in hiPSC-CMs, and that NKX2.5 deficiency-induced LD accumulation and CM apoptosis which can be rescued by LIPTER overexpression. Although the mechanism of hyperglycemia-repressed NKX2.5 remains to be investigated, our data suggest that the declined NKX2.5-LIPTER axis could possibility contribute to the progressive CM loss in diabetic heart. It is our hypothesis that hyperglycemia can differentially repress the NKX2.5-LIPTER expression in each individual CM of diabetic heart, and a minor amount of CMs with very low LIPTER expression level will gradually die and this slow progress of muscle loss will eventually lead to DCM of diabetic patients. Since intramyocyte LD accumulation precedes onset of cardiac dysfunctions in hearts of patients with obesity or diabetes, in vivo LIPTER transgene data strongly imply that delivery of LIPTER into heart muscle is an effective strategy to prevent cardiac dysfunction and heart failure of obese and diabetic patients.

1. A method of preserving cardiomyocyte function in a patient, said method comprising enhancing the concentration of LIPTER RNA in said cardiomyocytes (CMS).

2. The method of claim 1, wherein the method comprises increasing the expression of LIPTER RNA in said cardiomyocytes.

3. The method of claim 2, wherein exogenous nucleic acids encoding LIPTER RNA are introduced into said cardiomyocytes.

4. The method of claim 1, wherein the LIPTER RNA is an RNA comprising a sequence of SEQ ID NO: 1, SEQ ID NO: 2, SEQ ID NO: 3, SEQ ID NO: 4 and fragments thereof.

5. The method of claim 1, wherein the LIPTER RNA is an RNA comprising a sequence of SEQ ID NO: 4 or a fragment thereof.

6. The method of claim 4, wherein said exogenous nucleic acids are introduced into said cardiomyocytes via a viral vector, optionally wherein the viral vector is an adeno or adeno-associated viral vector.

7. The method of claim 4, wherein said exogenous nucleic acids are introduced into said cardiomyocytes via a nanoparticle transfection, wherein nucleic acids encoding LIPTER RNA are bound to the surface of the nanoparticle.

8. The method of claim 7, wherein said nanoparticle comprises phosphatidic acid (PA) and/or phosphatidylinositol 4-phosphate (PI4P).

9. The method of claim 1, wherein the concentration of LIPTER RNA in said cardiomyocytes is increased by the introduction of LIPTER RNA into said cardiomyocytes, optionally wherein the RNA is in the form of synthetic locked nucleic acids (LNA).

10. The method of claim 9, wherein the LIPTER RNA is an RNA comprising a sequence of SEQ ID NO: 1, SEQ ID NO: 2, SEQ ID NO: 3, SEQ ID NO: 4 and fragments thereof.

11. The method of claim 9, wherein the LIPTER RNA is an RNA comprising a sequence of SEQ ID NO: 4 or a fragment thereof.

12. The method of claim 1, wherein said patient is one who has lipid metabolism abnormality in the CMs of heart.

13. The method of claim 1, wherein said patient is one who has metabolic syndromes associated cardiac dysfunctions, cardiomyopathy and/or heart failure, including but not limited to diabetes and obesity.

14. The method of claim 1, wherein said patient is one who has cardiac dysfunctions, cardiomyopathy and/or heart failure.

15. (canceled)

16. (canceled)

17. A method of identifying subjects with abnormal lipid metabolism, said method comprising the steps of
measuring LIPTER expression levels in a test subject's cells;
comparing the detected LIPTER expression levels to the levels found in healthy subjects;
identifying a test subject with abnormal lipid metabolism when the detected LIPTER expression levels are lower than the levels found in healthy subjects.

18. The method of claim 17, wherein the LIPTER expression levels are measured by RT-qPCR, optionally using RNA recovered from cardiomyocytes of said test subject.

19. The method of claim 17, wherein a test subject is identified with abnormal lipid metabolism when the detected LIPTER expression levels are at least 5% lower than the levels found in healthy subjects.

20. A method of treating subjects identified as having abnormal lipid metabolism, said method comprising the steps of
measuring LIPTER expression levels in a test subject's cells;
comparing the detected LIPTER expression levels to the levels found in healthy subjects;
identifying a test subject with abnormal lipid metabolism when the detected LIPTER expression levels are lower than the levels found in healthy subjects
treating said identified test subjects using standard therapies for heart disease optionally including the administration of therapeutic agents to increase LIPTER RNA levels in the subject's cardiomyocytes.

21. The method of claim 20, wherein the subject is suffering from cardiac dysfunction associated with abnormal lipid metabolism of CMs, cardiomyopathy or heart failure.

22. The method of claim 21, further comprising improving heart function and Fatty Acid Oxidation (FAO) in the subject.
